(19) United States
(12) Patent Application Publication
(10) Pub. No.: US 2010/0273769 A1
(43) Pub. Date: Oct. 28, 2010

(54) COMPOSITION AND METHOD FOR THE TREATMENT OF PARKINSON'S DISEASE

(76) Inventor: Hanno RODER, West Jacksonville, FL (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/693,898

(22) Filed: Jan. 26, 2010

Related U.S. Application Data
(60) Provisional application No. 61/206,078, filed on Jan. 26, 2009.

(51) Int. Cl. A61K 31/553; A61P 25/16

(52) U.S. Cl. 514/211.08

(57) ABSTRACT

The present invention relates to the use of specific indolocarbazole compounds for the preparation of pharmaceutical compositions for the treatment of Parkinson's disease. In particular, the compounds of the invention are useful for the prevention or treatment of PD, similar forms of Parkinsonism, and synucleopathies involving Lewy body neurodegeneration.

### RELATED APPLICATIONS

This application claims priority to U.S. Provisional Application No. 61/206,078, filed Jan. 26, 2009, titled “COMPOSITIONS AND METHOD FOR THE TREATMENT OF PARKINSON'S DISEASE.” The contents of any patents, patent applications, and references cited throughout this specification are hereby incorporated by reference in their entireties.

### FIELD OF THE INVENTION

The present invention relates to the use of specific indolocarbazole compounds for the preparation of pharmaceutical compositions for the treatment of Parkinson's disease.

### BACKGROUND OF THE INVENTION

Parkinson's disease (also known as Parkinson disease or PD) is a degenerative disorder of the central nervous. PD often impairs the sufferer's motor skills, speech, and other functions. It is characterized by (1) a slowing down of all movements (bradykinesia), quiet and monotonous speech (akinesia or hypokinesia), absence of the physiological associated movements, a stooped posture, a small-step, partially shuffling gait, handwriting which becomes smaller as the writing continues, uncontrollable disturbances in movement, with a tendency to fall forward to the side or backward, (2) rigidity of the musculature (rigor), and (3) coarse resting tremor (trembling). The primary symptoms of PD are the result of decreased stimulation of the motor cortex by the basal ganglia, normally caused by the insufficient formation and action of dopamine, which is produced in the dopaminergic neurons of the brain. Secondary symptoms may include high level cognitive dysfunction and subtle language problems. PD is both chronic and progressive.

Parkinson's disease is the second most common neurodegenerative disorder, after Alzheimer's disease. Parkinson's disease has a prevalence of approximately 0.5 to 1 percent among persons 65 to 69 years of age, rising to 1 to 3 percent among persons 80 years of age and older (see Tanner et al., Neurol Clin. 14, 317-335 (1996)).

Accordingly, there remains a need for effective therapies for Parkinson's disease.

### SUMMARY OF THE INVENTION

The invention provides compounds, pharmaceutical compositions comprising such compounds and methods of using such compounds to treat or prevent diseases or disorders associated with Parkinson's disease, as well as any a synucleopathy involving Lewy body neurodegeneration.

Thus, in one aspect provided herein is a compound of general formula 1, or a pharmaceutically acceptable salt thereof,

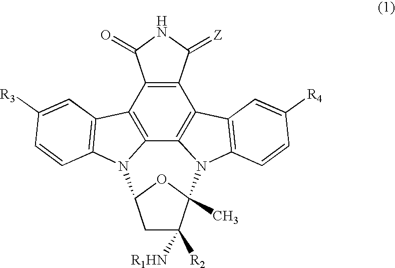

wherein variables R₁-R₄ are as defined below.

In one embodiment, proved herein are compounds of the formula 1, having the formulas 2-5, both in the absolute and relative stereochemical configuration corresponding to the natural product (+)-K252a:

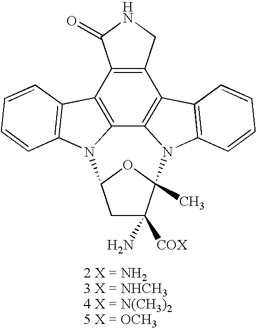

In another aspect, provided herein is a method for the prevention or treatment of Parkinson's Disease in a subject, comprising administering to a subject in need thereof an effective amount of an indolocarbazole compound of the general formula 1 or a pharmaceutically acceptable salt thereof.

In still another aspect, provided herein is a method for the prevention or treatment of a synucleopathy involving Lewy body neurodegeneration, comprising administering to a subject in need thereof an effective amount of an indolocarbazole compound of the general formula 1, or a pharmaceutically acceptable salt thereof. In one embodiment, the synucleopathy involving Lewy body neurodegeneration is idiopathic PD, Dementia with Lewy Bodies (DLB), or familial PD caused by mutations in LRRK2, SNCA (alpha-synuclein), UCHL-1 (ubiquitin carboxyl-terminal hydrolase L1), PRKN (parkin), or PINK-1 (PTEN-induced putative kinase).

In another embodiment, provided herein is the use of an inolocarbazole compound of the general formula 1, or a pharmaceutically acceptable salt thereof, for the preparation of a pharmaceutical composition for the prevention or treatment of PD, similar forms of Parkinsonism, and synucleopathies involving Lewy body neurodegeneration.

In another aspect, provided herein is a use of a compound of the general formula 1 for the manufacture of a medicament for the prevention or treatment of Parkinson's Disease in a subject in need thereof. In still another aspect, provided herein is a use of a compound of the general formula 1 for the manufacture of a medicament for the prevention or treatment of a synucleopathy involving Lewy body neurodegeneration.

In another aspect, provided herein is the use of a compound of the general formula 1 for the manufacture of a pharmaceutical for the prevention or treatment of Parkinson's Disease in a subject in need thereof. In yet another aspect, provided herein is the use of a compound of the general formula 1 for the manufacture of a pharmaceutical for the prevention or treatment of a synucleopathy involving Lewy body neurodegeneration in a subject in need thereof.

In another aspect, provided herein is a method of inhibiting the activity of wildtype LRRK2 or mutant LRRK2, comprising utilizing a compound of formula 1. In yet another embodiment, provided herein is a method of treating a disease in a subject, wherein the disease etiology or progression is at least partially mediated by the activity of wildtype LRRK2 or mutant LRRK2, comprising administering to the subject a compound of formula 1.

### BRIEF DESCRIPTION OF THE DRAWINGS

FIG. 1: Schematic Representation of mutations of human LRRK2 causing PD within the domain structure of the kinase.

FIG. 2: Estimated kinase specificity spectrum of the representative compound 3 obtained by extrapolation of in vitro kinase inhibition potencies extrapolated to physiological ATP concentrations (2-3 mM) in view of its generally ATP-competitive mechanism of action. The dotted line indicates the solubility limit in water at neutral pH delineating those kinases where absent other factors (e.g. concentration of kinase in vivo) a partial or complete inhibition is clinically feasible (red).

FIG. 3: Inhibitory curves obtained for wild-type LRRK2 (A) and the disease-causing mutant G2019S LRRK2 (B) with the representative compound 3 at 100 μM ATP using the Adapta® assay system (Invitrogen), with IC₅₀ curve transition highlighted in green. Note the IC₅₀ values substantially below the nominal concentration of the kinase itself.

FIG. 4: Loss of cephalic dopaminergic neurons in wild-type (closed triangles) and mutant (closed squares) LRRK2 transgenic worms during adult stages day 0-9 relative to GFP control worms (closed circles) and a kinase-dead LRRK2 mutant worm line (open circles).

FIG. 5: Age-dependent loss of the motility slowing response to food relative to lack thereof induced by active LRRK2 proteins (wild-type: closed triangle; R1441C: closed square, but not by a kinase-inactive form of LRRK2 (open circles). The loss of the food-sensing response can be partially rescued in the impaired mutants by addition of 2 mM dopamine hydrochloride to the assay (inset).

FIG. 6: Prevention of the loss of the food-sensing response in the R1441C LRRK2 mutant by the presence of increasing concentrations of the representative compound 3 during worm development to adult stage day 4. The dotted line indicates the control food-sensing response in GFP-transgenic worms lacking LRRK2 expression.

### DETAILED DESCRIPTION OF THE INVENTION

Parkinson's Disease (PD) is a movement disorder characterized by gradually progressing bradykinesia, resting tremor, and postural instability with an age-related onset [Gelb et al., Arch. Neurol. 56, 33-39 (1999)]. In its typical manifestation, it involves primarily the degeneration and loss of dopaminergic neurons in the substantia nigra, resulting eventually in severe deficiency of the neurotransmitter dopamine. This type of neurodegeneration involves the formation of intracellular inclusion bodies (Lewy bodies) [Formo, J. Neuropathol. Exp. Neurol. 55, 259-272 (1996)], which contain the protein synuclein as a major constituent [Spillantini et al., Nature 388, 839-840 (1997); Baba et al., Am. J. Pathol. 152, 879-884 (1998)]. PD can therefore be classified as a distinct protein aggregation disorder, like Alzheimer's disease, prion diseases, or Huntington's disease, but with a fundamentally different pathobiochemistry affecting more specific subpopulations of neurons. Possibly a combination of specific properties of substantia nigra neurons, like being dopaminergic and containing neuromelanin iron stores (the source of the unique dark pigmentation of these neurons), may provide for selective vulnerabilities in combination with other factors.

Besides classical PD, Parkinsonism-related disorders have been defined with similar impairment of movement as in PD, but extended symptomatology involving also memory and cognitive functions. In such cases Lewy body formation has spread to cortical areas as well, providing for considerable diagnostic overlap with Dementia with Lewy bodies (DLB). Because of the pervasive involvement of synuclein in Lewy body formation, these diverse disorders are grouped under the term Synucleopathies. In spite of this conspicuous association, however, Lewy bodies may be more of a classification feature, reporting a specific pathobiochemistry, rather than a direct cause of neurodegeneration [Jellinger, Biochem. Biophys, Acta 2008; Parkinnen et al., Acta Neuropathol. 116, 125-128 (2008)]. On the other hand, the observed commonalities do suggest that certain forms of Parkinson's Disease with Dementia (PDD) are mechanistically related to classical PD. However, there are also forms of PDD with completely unrelated disease biology involving a different form of neurodegeneration based on the pathobiochemistry of the microtubule-associated protein tau (tauopathy), as most clearly exemplified by Frontotemporal Dementia with Parkinsonism caused by mutations in tau protein on chromosome 17 (FTDP-17) [Hutton et al., Nature 393, 702-705 (1998)]. Hence, in view of the evolving molecular insights into the basis of these neurological disorders the classical clinical diagnoses will become more advantageously replaced by disease-mechanism based classifications, especially if the therapeutic consequences of diagnosis are increasingly less oriented on symptom relief but rather on causative treatment strategies. A clear example is provided by the distinction between synucleopathies (most forms of PD) and tauopathies (e.g. Alzheimer's disease, certain forms of frontotemporal dementia, Pick's disease, progressive supranuclear palsy (PSP), corticobasal degeneration (CBD).

The majority of PD does not have a single apparent cause (sporadic or idiopathic PD), in that both environmental factors and genetic susceptibilities may play a role. Specifically for substantia nigra degeneration, the age-related decay of the integrity of mitochondrial DNA (mtDNA), encoding for vital respiratory chain subunits, has been invoked as a metabolic driver of neurodegeneration [Bender et al., Nat. Genet. 38, 515-517 (2006); Vila et al., J. Neurochem. 107, 317-328 (2008)]. It is thought that heterogeneous mtDNA damage is accumulated over time by exposure to oxygen radicals, possibly formed preferentially in substantia nigra neurons due to their prominent iron content. This provides the rationale for using the mitochondrial toxin MPTP in animal models to selectively destroy dopaminergic neurons in the substantia nigra and striatum.

More recently, various genetic factors have been identified in those instances where PD is clearly inherited, altogether comprising about 10% of all cases. Most significantly in view of the defining neuropathology of Lewy bodies, mutations in the synuclein gene were identified as a cause of PD [Polymeropoulos et al., Science 276, 2045-2047 (1997)]. Since then mutations in a range of other genes were linked to familial PD cases [Tan et al., Hum. Mutat. 28, 641-653 (2007)], all of them displaying dysfunctions of mitochondria, oxidative stress, and protein aggregation in Lewy bodies, as in idiopathic PD.

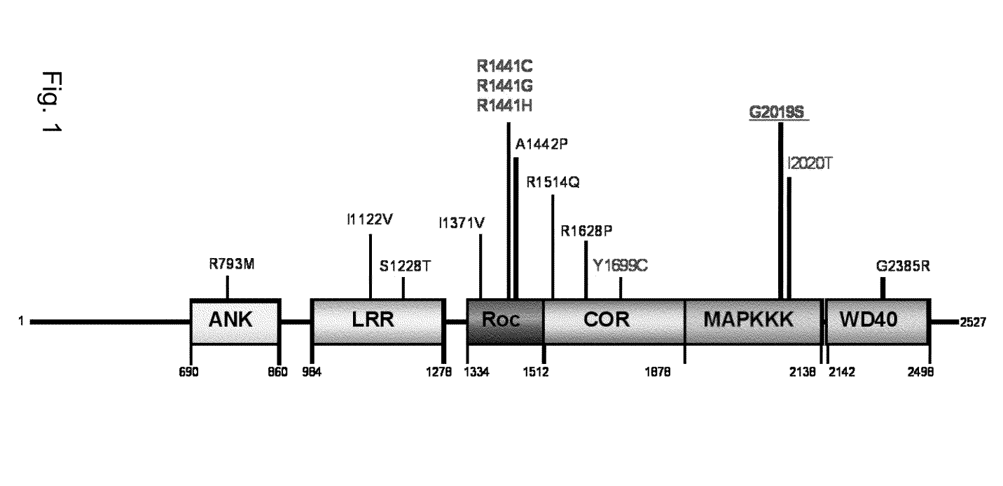

Identification of mutations in the leucine-rich repeat kinase 2 gene (LRRK2) has been reported as the most frequent cause of autosomal dominantly inherited PD [Paisan-Ruiz et al., Neuron 44, 595-600 (2004)], comprising about 1-2% of all unambiguously familial PD cases (FIG. 1). However, it has also been reported that the impact of this gene may also extend beyond the clearly familial PD cases, as mutations with moderate penetrance have been identified in patients that only seem to be sporadic due to insufficient extend of family history and late onset [Bonifati, Neurochem. Res. 32, 1700-1708 (2007); Latourelle et al., BMC Med. 6, 32 (2008)]. Several mutations have been shown to increase its kinase activity directly or indirectly [Lu et al., J. Neurosci. Res. 86, 1895-1901 (2008)], providing a straightforward rationale for inhibition of this kinase as a causal therapeutic modality for PD and synucleopathies in general, similar to inhibition of dysregulated specific kinase activities due to oncogenic kinase mutations in cancer. The pathomechanistic relevance of elevated kinase activity of LRRK2 is supported by cell culture studies, where the cytotoxicity as well as the neurite growth inhibition induced by pathogenic LRRK2 mutants is antagonized by the simultaneous introduction of a kinase-dead variant [Smith et al., Nat. Neurosci. 9, 1231-1233 (2006); MacLeod et al., Neuron 52, 587-593 (2006)].

The G2019S mutation of LRRK2 is of particular pathomechanistic relevance as it directly increases kinase activity several-fold, and is at the same time the most prevalent genetic cause in unselected PD populations, detectable in 5-10% of all late onset PD cases [Bonifati, Neurochem. Res. 32, 1700-1708 (2007); Infante et al., Neurosci. Lett. 395, 224-226 (2005); Gorostidi et al., Neurogenetics 2008; Gaig et al., Arch. Neurol. 63, 377-382 (2006); Ferreira et al., Mov. Disord. 22, 1194-1201 (2007)].

Recent functional genetics analyses in simple model organisms suggest that mutant LRRK2 leads to a more general upregulation of protein translation by activating the translation initiation factor eIF4E, which is associated with enhanced susceptibility to cellular stress and mitochondrial damage, long recognized as a feature of PD-specific neurodegeneration [Imay et al., EMBO J. 27, 2432-2443 (2008)]. The fact that other PD-related genes, like PARK2 and PINK1, also activate eIF4E provides a potential common framework for a pathomechanism of PD centered around cellular stress [Tain et al., Nat. Neurosci. 12, 1129-1135 (2009)]. An inherent feature of this concept is that LRRK2 activity can participate in pathogenic events even if not mutated by virtue of adding to an overactivation of the pathway already burdened by other inputs, as suggested by the finding that deletion of the endogenous LRRK2 homolog in genetic model organisms antagonizes the neurodegenerative activity of PARK2 and PINK1 [Tain et al., Nat. Neurosci. 12, 1129-1135 (2009)]. Conversely, the incomplete penetrance of LRRK2 mutations can be explained seamlessly by a compensatory reduction of input from parallel pathways. On a clinical level, the operation of a common pathway is reflected in the fact that idiopathic PD and PD caused by the various genes identified to date are essentially not distinguishable. It is therefore likely that inhibition of LRRK2 is a therapeutic concept not limited to patients with LRRK2 mutations. This assertion is strongly supported by transgenic mouse studies examining the interaction between LRRK2 and disease-causing mutations of synuclein, another PD-related gene. Overexpression of wild-type human LRRK2 greatly accelerated synuclein pathology and associated neurodegeneration in mice already transgenic with the disease-causing human mutant A53T α-synuclein, while suppression of LRRK2 expression using an inducible promoter ameliorated the A53T mutant synuclein phenotype [Lin et al., Neuron 64, 807-827 (2009)].

Thus, provided herein are compounds of the general formula 1 or a pharmaceutically acceptable salt thereof:

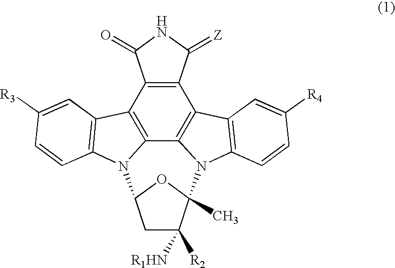

wherein:

R₁ is H, methyl, ethyl, iso-propyl, or acetyl;

R₂ is COOR₅, wherein R₅ is H, methyl, ethyl, isopropyl, cyclopropyl, or —(CH₂)ₙ—Y, wherein Y is OR′, NR′R″, N-morpholinyl, N-piperazinyl, or N′-alkyl-N-piperazinyl, wherein R′ and R″ are independently H or lower n-alkyl and n is 2-6;

or R₂ is CONR₅R₆, wherein R₅ and R₆ are independently H, methyl, ethyl, isopropyl, cyclopropyl, or —(CH₂)ₙ—Y, wherein Y is OR′, NR′R″, N-morpholinyl, N-piperazinyl, or N′-alkyl-N-piperazinyl, wherein R′ and R″ are H or lower n-alkyl, and n is 2-6), or R₅ is any of these definitions, and R₆ is CH₃—(CH₂)ₙ—CO, Ph-CO, or CF₃—CO, wherein n is 0-5;

or R₂ is COX, wherein X is N-morpholinyl, N-piperazinyl, or N′-alkyl-N-piperazinyl;

or R₂ is CH₂OR₅, wherein R₅ is H, methyl, ethyl, isopropyl, cyclopropyl, —(CH₂)ₙ—X, CH₃—(CH₂)ₘ—CO, Ph-CO, or CF₃—CO, wherein X is OR′, NR′R″, N-morpholinyl, N-piperazinyl, or N′-alkyl-N-piperazinyl, wherein R′ and R″ are independently H or lower n-alkyl, n is 2-6, and m is 0-5;

or R₂ is CH₂NR₅R₆, wherein R₅ and R₆ are independently H, methyl, ethyl, isopropyl, cyclopropyl, —(CH₂)ₙ—Y, Ph-CO, or CF₃—CO, wherein Y is OR′, NR′R″, N-morpholinyl, N-piperazinyl, or N′-alkyl-N-piperazinyl, R′ and R″ are independently H or lower n-alkyl, and n is 2-6, or R₅ is any of these definitions, and R₆ is CH₃—(CH₂)ₙ—CO, wherein n is 0-5;

R₃ and R₄ are independently H, F, Cl, Br, methyl, ethyl, propyl, iso-propyl, n-alkyl, OH, OCH₃, O(CH₂)ₙCH₃ (n is 1-6), OCH(CH₃)₂, OC(CH₃)₃, CH₃(CH₂)ₙCO (n is 0-5), CF₃—CO, CH₃(CH₂)ₙCOO (n is 0-5), CF₃—COO, or NR₅R₆, wherein R₅ and R₆ are independently H, methyl, ethyl, propyl, iso-propyl, or n-alkyl, or R₅ is any of these definitions, and R₆ is CH₃—CO or CF₃—CO);

or one or both of R₃ and R₄ are independently CH₂—X—(CH₂)ₙCH₃, wherein X═O, S, and n is 0-5;

or one or both of R₃ and R₄ are independently CH₂—NR₅R₆, wherein R₅ and R₆ are independently H, methyl, ethyl, propyl, iso-propyl, n-alkyl, or n-acyl, or R₅ is any of these definitions, and R₆ is CH₃—CO or CF₃—CO); and

Z is either (H, H) or O.

In one embodiment of formula 1, R₁ is H, methyl, ethyl, iso-propyl, or acetyl;

R₂ is CH₂OR₅, wherein R₅ is H, methyl, ethyl, isopropyl, cyclopropyl, —(CH₂)ₙ—X, CH₃—(CH₂)ₘ—CO, Ph-CO, or CF₃—CO, wherein X is OR′, NR′R″, N-morpholinyl, N-piperazinyl, or N′-alkyl-N-piperazinyl, R′ and R″ are H or lower n-alkyl, n is 2-6, and m is 0-5;

or R₂ is CH₂NR₅R₆, wherein R₅ and R₆ are independently H, methyl, ethyl, isopropyl, cyclopropyl, or —(CH₂)ₙ—Y, wherein Y is OR′, NR′R″, N-morpholinyl, N-piperazinyl, or N′-alkyl-N-piperazinyl, wherein R′ and R″ are independently H or lower n-alkyl and n is 2-6, or R₅ is any of these definitions, and R₆ is CH₃—(CH₂)ₙ—CO, Ph-CO, or CF₃—CO, wherein n=0-5;

R₃ and R₄ are independently H, F, Cl, Br, methyl, ethyl, propyl, iso-propyl, n-alkyl, OH, OCH₃, O(CH₅)ₙCH₃ (n is 1-6), OCH(CH₃)₂, OC(CH₃)₃, CH₃(CH₂)ₙCO (n is 0-5), CF₃—CO, CH₃(CH₂)ₙCOO (n is 0-5), CF₃—COO, or NR₅R₆, wherein R₅ and R₆ are independently H, methyl, ethyl, propyl, iso-propyl, or n-alkyl, or R₅ is any of these definitions, and R₆ is CH₃—CO or CF₃—CO;

or one or both of R₃ and R₄ are CH₂—X—(CH₂)ₙCH₃, wherein X is O or S, and n is 0-5;

or one or both of R₃ and R₄ are CH₂—NR₅R₆, wherein R₅ and R₆ are independently H, methyl, ethyl, propyl, iso-propyl, n-alkyl, or n-acyl, or R₅ is any of these definitions, and R₆ is CH₃—CO or CF₃—CO), and

Z is (H, H) or O.

In another embodiment of formula 1, Z is (H, H). In another embodiment, R₃ and R₄ are H. In still another embodiment, R₁ is H. In yet another embodiment, R₂ is CONR₅R₆, wherein R₅ and R₆ are each independently H, methyl, ethyl, or isopropyl, or is R₂ is COOR₅, wherein R₅ is H, methyl, ethyl, or isopropyl.

In another embodiment of formula 1, R₁ is H, and R₂ is CH₂OH, CH₂OCH₃, or CH₂NR₅R₆, wherein R₅ and R₆ are independently H or methyl, and R₃ and R₄ are H.

In a particular embodiment, the compounds of formula 1 are are compounds of the formulas 2-5, both in the absolute and relative stereochemical configuration corresponding to the natural product (+)-K252a:

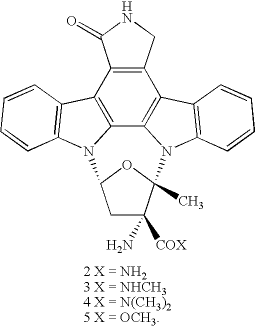

Thus, in one embodiment, provided herein is a method of treating PD in a subject in need thereof, comprising administering to the subject compound 2, such that the PD is treated. In another embodiment, provided herein is a method of treating PD in a subject in need thereof, comprising administering to the subject compound 3, such that the PD is treated. In another embodiment, provided herein is a method of treating PD in a subject in need thereof, comprising administering to the subject compound 4, such that the PD is treated. In another embodiment, provided herein is a method of treating PD in a subject in need thereof, comprising administering to the subject compound 5, such that the PD is treated.

Compounds of the general formula 1 had previously been disclosed for uses relating to cancer and for immune suppression (WO 97/05140, incorporated herein by reference in its entirety), and for the specific use of inhibiting the hyperphosphorylation of the microtubule-associated protein tau and thereby the process of neurofibrillary degeneration which characterizes the distinct type of neurodegeneration in Alzheimer's disease and other tauopathies (WO 05/117550).

Applicant has unexpectedly discovered that compounds of the general formula 1, e.g., the derivatives of the formulas 2-5, are unusually potent inhibitors of wild-type LRRK2 and of the G2019S mutant of LRRK2. This finding could not be anticipated from the utility of these compounds for tau-driven neurodegeneration, since the pathomechanisms of tauopathies in various dementias are fundamentally unrelated to the synucleopathy as expressed in classical PD, and the various genetic causes of these two pathologies do not overlap. In particular, no enrichment of LRRK2 mutant carriers was found in populations of Alzheimer's disease or the tauopathy Progressive Supranuclear Palsy (PSP) [Toft et al., Mech. Ageing Dev. 126, 1201-1205 (2005); Lee et al., Am. J. Med. Genet. B Neuropsychiatr. Genet. 141B, 549-550 (2006); Tedde et al., Cell. Mol. Neurobiol. 27, 877-881 (2007); Ross et al., Neuropathol. Appl. Neurobiol. 32, 23-25 (2006); Madzar et al., Eur. J. Neurol. 16, 1230-1232 (2009)]. It has been reported that two generic representatives of the compound class, staurosporine and K252a were active as inhibitors of LRRK2 [Covy and Giasson, Biochem. Biophys. Res. Commun. 378, 473-477 (2009); Anand et al., FEBS J. 276, 466-478 (2009)], however, compounds of the general formula 1, e.g., compounds 2-5, displayed a surprisingly much higher potency than K252a, and even a higher potency as well as a much more restricted selectivity profile than staurosporine. The potency of K252a for LRRK2 inhibition in a direct comparison is more than 2 orders of magnitude lower than that of the most closely related compound of the invention 5 (Table 1), the two compounds being only distinguished by the presence of an —OH group (K252a) and a —NH₂ (5) group at the glycone moiety, respectively. Unlike for a spectrum of other less potently inhibited kinases (Table 1), tau hyperphosphorylation (WO 05/117550, incorporated herein by reference in its entirety), and inhibition of tumor cells (WO 08/076,394, incorporated herein by reference in its entirety), the carboxamide functionality (R₂═CONHR in the general formula 1) geminal to the amino group is not essential to potent LRRK2 inhibition (Table 1), allowing for significantly enhanced specificity for LRRK2 inhibition. Kinase specificity within the scope of compounds of the general formula 1 is further enhanced by the compatibility of substituents R₃ and R₄ other than H with potent LRRK2 inhibition, which is not the case with many other kinases.

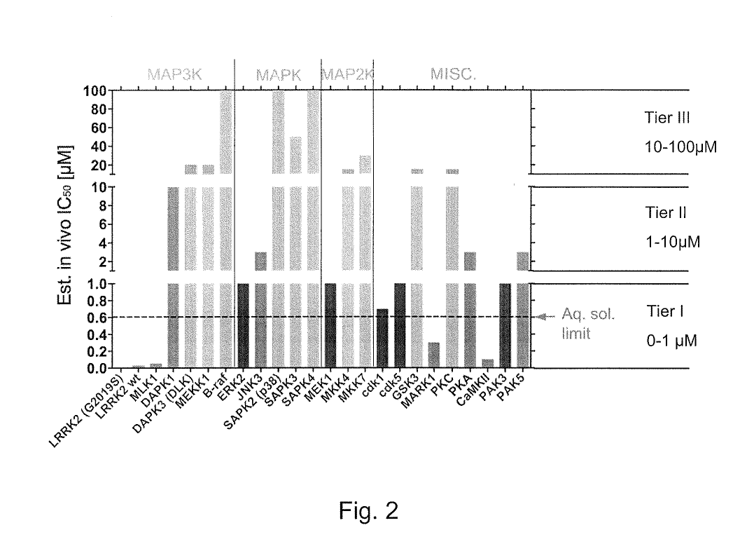

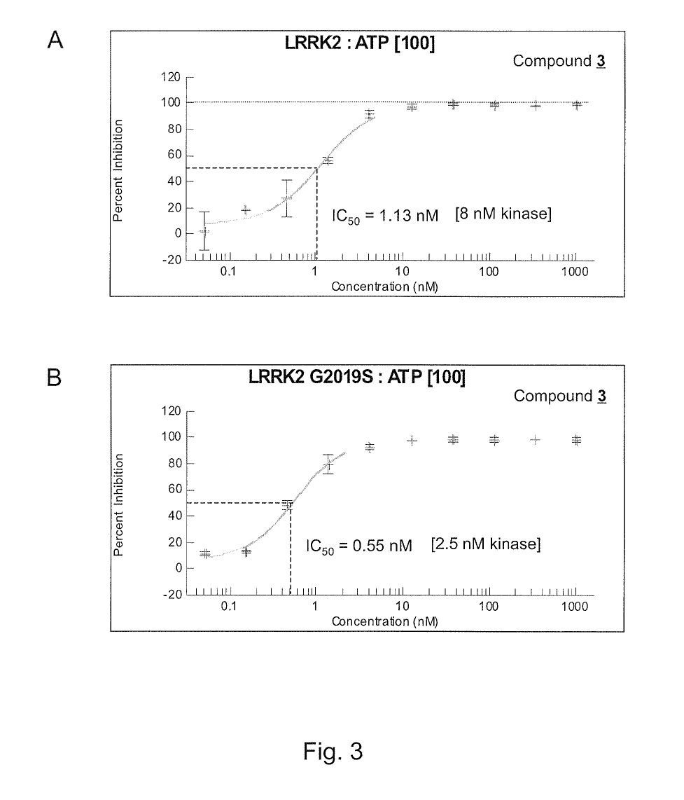

The most significant aspect of the current invention is therefore the unexpected realization of the decisive impact of specifically the amino-group to confer useful potency relative to the corresponding OH group in the natural scaffold of K252a derived compounds. In fact, LRRK2 is the most potently inhibited kinase by the derivatives of the formulas 2-5 in a set of over 20 representative kinases, with a 10 to 100,000 fold selectivity over other members of the kinase panel (FIG. 2), a preference not reflected in other members of the compound class and not suggested by prior art. At 100 microM concentration of the co-substrate ATP half-maximal inhibition was achieved at or below a nominal concentration of 1 nanomolar (FIG. 3), but being essentially limited by the concentration of the kinase itself in the assay. Although compounds of the general formula 1 are competitive with ATP, and concentrations of ATP in vivo are 20-30 fold higher than in the in vitro assay, compounds of the general formula 1, e.g., compounds of the formulas 2-5, are effective to reduce LRRK2 kinase activity in vivo significantly at concentrations around 10-30 nanomolar. This inhibitory potency is 2-3 orders of magnitude higher than for kinases B-raf, MEKK1, MEK1/2, MKK4, MKK7, ERK2, JNK1, cdk1, cdk5, MARK1, GSK3, PAK3, PAK5, PKC, PKA, and about one order of magnitude more potent than inhibition of the kinase MLK1 under comparable conditions (FIG. 2). This degree of potency and selectivity is essential for the therapeutic utility of compounds of the general formula 1 in view of the generally very low water solubility of compounds of the indolocarbazole class. Any useful potency must be below the limit of aqueous solubility even at the rather high ATP concentrations in vivo, which is only the case for LRRK2 but not for most other kinases, establishing an even more robust differentiation of LRRK2 from other kinases than the in vitro kinase assay profile might suggest prima facie (FIG. 2). In contrast, staurosporine inhibits at least cdks, PKC, PKA, and MARKs at potencies comparable to LRRK2, while at the same time the potency of most other analogous structures based on the natural staurosporine and K252a scaffolds is too low in relation to their solubility to be therapeutically irrelevant in any realistic clinical application scheme.

Compounds of the general formula 1 are believed to be the most potent inhibitors of LRRK2 discovered to date. For example, under identical assay conditions compounds 3 and 5 are significantly more potent than staurosporine, the most potent compound disclosed in the prior art [Anand et al., FEBS J. 276, 466-478 (2009)] (5 nanomolar at 100 microM ATP vs. essentially receptor-limited potency of the compounds of the invention, i.e. ≦1 nM). The potency notwithstanding, compound 3 for example has a wide therapeutic index, in that plasma concentrations of 600-800 nanomolar can be sustained for at least one month in rodents without detectable toxicity, while 200-300 nanomolar plasma concentrations of staurosporine are the LC₅₀ in a single dosing. Therefore, unlike prior art compounds of the indolocarbazole class, the compounds of this invention represent clinically useful therapeutic agents by inhibiting, within their aqueous solubility limits, LRRK2 in a chronically tolerable way.

Compounds of the general formula 1, but especially compounds 2-5, are orally bioavailable in solid dosage forms, and achieve sustained brain concentrations between 300 and 500 nanomolar, more than sufficient for significant inhibition of LRRK2, can be sustained chronically with once or twice daily application of 10 mg/kg p.o. without incurring prohibitive side effects, as disclosed in WO 08/076,394, herein incorporated by reference. Hence compounds of the general formula 1, but especially compounds 2-5, are useful to reduce or suppress aberrantly activated LRRK2 in patients predisposed to or suffering from PD or similar synucleopathies, in order to slow or halt the progression of neurodegeneration driven by this specific pathology, and of the commensurate debilitating symptoms in such patients.

### Methods of Preparation

Compounds 2-5 can be prepared as disclosed in WO 08/076,394 (incorporated herein by reference in its entirety). Other compounds of the general formula 1 can be prepared from the compound of the formula 5 (Scheme 1), after protection of the lactam moiety by acetylation with acetic anhydride or trifluoroacetic anhydride in pyridine or a similar basic solvent, using methods to introduce various substituents R₃ and R₄ as extensively disclosed in the prior art, e.g. in WO 88/07045 (herein incorporated in its entirety by reference). R₁ substituents of compounds of the general formula 1 can be introduced into protected compounds 6 by alkylation (e.g. methyl iodide in the presence of a base) or acylation using acylchlorides in the presence of pyridine or triethylamine as described in WO 97/05140. Protected Esters 6 can then be converted into amides 7 by KCN-catalyzed aminolysis (as disclosed in WO 08/076,394) with the desired amine, or can be converted into the corresponding alcohols 10 by reduction with sodium borohydride or lithium aluminum hydride, or into an ether 8 with triethylsilane in the presence of the respective alcohol and indium tribromide [Sakai et al., J. Org. Chem. 72, 5920-5922 (2007)]. Alcohols 10 can be further derivatized to amines 12 by nucleophilic substitution of the tosylate 11 with the appropriate amine in aprotic polar solvents and in the presence of a sterically hindered base like diazabicyclo-undecene (DBU) [WO 88/07045, incorporated herein by reference in its entirety]. It is evident to those skilled in the art that the conversion of several functional groups can be effected by sequential application of the transformations of Scheme 1. Final deprotection of intermediates to form active compounds of the general formula 1 is generally accomplished with aqueous

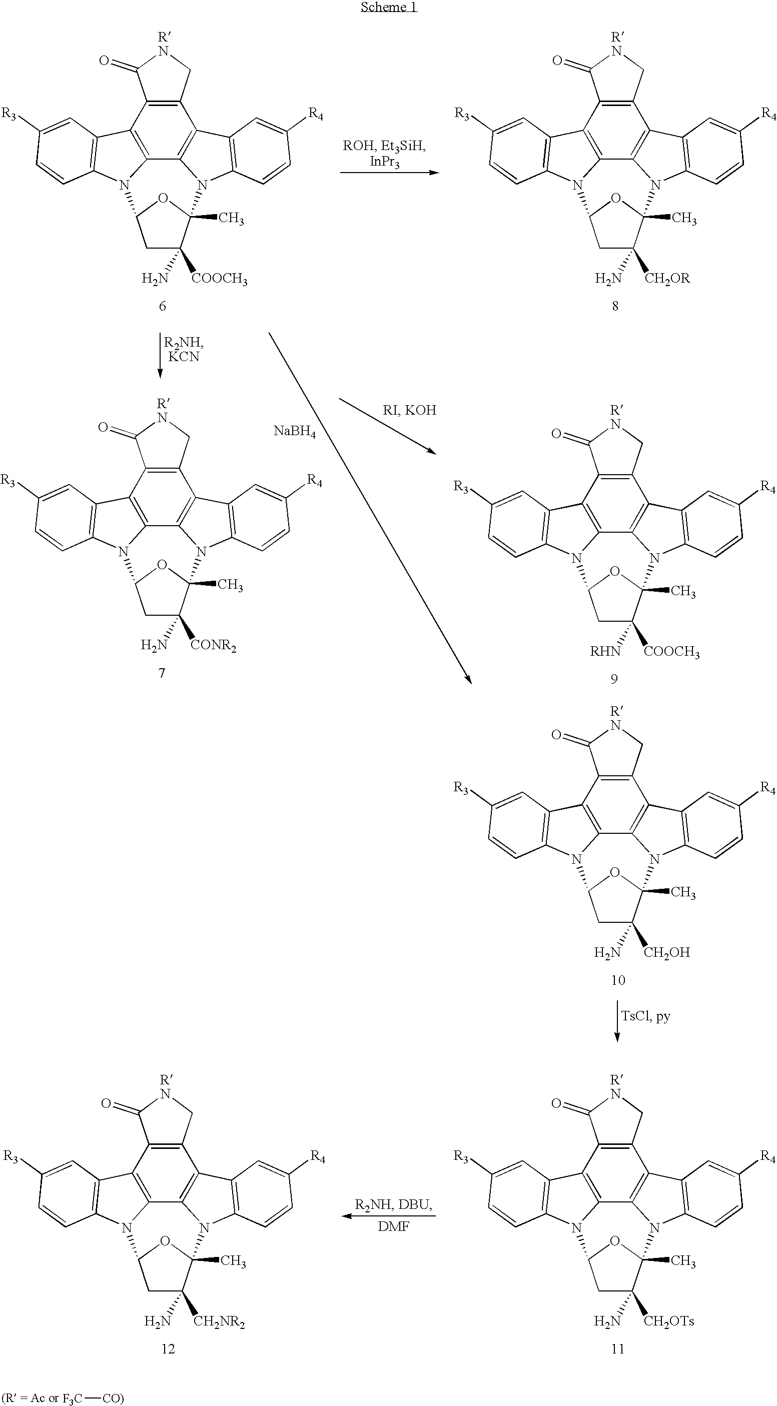

potassium hydroxide, or sodium methoxide in ether solvents if the intermediates were protected by acetylation. These methods of preparation should not be construed to limit the scope of the disclosure. Alternative methods to access the structures within the scope of the invention may be apparent to those skilled in the art.

The compounds of general formula 1 can form salts that are also within the scope of this invention. Reference to a compound of general formula 1 herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of general formula 1 contains both a basic moiety, such as, but not limited to an amino group, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable salts) are preferred. Salts of the compounds of the general formula 1 may be formed, for example, by reacting a compound of general formula 1 with an amount of acid or base, such as an equivalent amount, in a medium such as one in which the salt precipitates (e.g. THF, dioxane, or ethyl ether). Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include benzenesulfonates, bisulfates, dodecylsulfates, ethanesulfonates, glycerophosphates, hemisulfates, hydrochlorides, hydrobromides, hydroiodides, 2-hydroxyethanesulfonates, methanesulfonates, methyl sulfates, 2-naphthalenesulfonates, nitrates, persulfates, phosphates, sulfates, sulfonates (such as those mentioned herein), toluenesulfonates (also known as tosylates), and the like.

All such acid and base salts are intended to be pharmaceutically acceptable salts within the scope of the invention and all acid and base salts are considered equivalent to the free forms of the corresponding compounds for purposes of the invention.

Compounds of the general formula 1 exist in different isomeric (e.g., enantiomers, diastereoisomers, atropisomers) forms. The active compounds of this invention have the absolute and relative configuration of the natural product (+)-K252a.

Polymorphic forms of the compounds of the general formula 1, and of the salts, solvates and prodrugs of the compounds of general formula 1, are intended to be included in the present invention.

### Methods of Treatment

This invention provides a method for inhibiting the activity of the kinase LRRK2 or any of its mutant forms, by administering an effective amount (e.g., a therapeutically effective amount) of one or more (e.g., one) compounds of the formulas 1 and 2-5. This reduces either the activity of LRRK2 in the absence of mutations, which may be activated by environmental stimuli or engages excessively in its pathway due to reduced antagonist activity, or which reduces the aberrantly elevated or otherwise dysregulated activity of LRRK2 caused by disease-associated mutations.

This invention also provides a method for preventing or delaying the progression of the neurodegeneration in synucleopathies related to abnormal LRRK2 activity by administering an effective amount (e.g., a therapeutically effective amount) of one or more (e.g., one) compounds of formula 1, and 2-5 to a patient in need of such treatment. In another embodiment, this invention provides a method for preventing or delaying the progression of the neurodegeneration in synucleopathies caused by a mutation in LRRK2 by the administration of an effective amount (e.g., a therapeutically effective amount) of one or more (e.g., one) compounds of formula 1 and 2-5.

Examples of synucleopathies (or Lewy body diseases) that may be prevented or treated include, but are not limited to: idiopathic PD, familial PD caused by mutations in LRRK2, SNCA (alpha-synuclein), UCHL-1 (ubiquitin carboxyl-terminal hydrolase L1), PRKN (parkin), PINK-1 (PTEN-induced putative kinase), Dementia with Lewy Bodies (DLB). In a preferred embodiment, the subject to be treated is human. In another preferred embodiment, the subject to be treated suffers from idiopathic PD. In yet another preferred embodiment, the subject to be treated is a carrier of a mutation of LRRK2 associated with PD, including but not limited to those listed in FIG. 1. In an especially preferred embodiment the subject is heterozygous for the G2019S mutation of LRRK2.

The term “treat,” “treated,” “treating” or “treatment” includes the diminishment, amelioration, or alleviation of at least one symptom associated with or caused by the state, disorder or disease being treated, e.g., PD, similar forms of Parkinsonism, and synucleopathies involving Lewy body neurodegeneration. In certain embodiments, the treatment comprises the induction of PD or a PD-associated disorder, followed by the activation of the compound of the invention, which would in turn diminish or alleviate at least one symptom associated or caused by the PD or a PD-associated disorder being treated. Treatment can be diminishment of one or several symptoms of a disorder or complete eradication of a disorder.

The term “subject” is intended to include organisms, e.g., prokaryotes and eukaryotes, which are capable of suffering from or afflicted with a disease, disorder or condition associated with the activity of a protein kinase. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from PD, similar forms of Parkinsonism, and synucleopathies involving Lewy body neurodegeneration. In another embodiment, the subject is a cell.

### Methods of Administration

The compounds of formula 1, e.g., compounds 2-5, can be administered orally, preferably as a solid dosage form, more preferably a capsule, and while the total therapeutically effective daily dose can be administered in one to four, or one to two divided doses per day, generally, the therapeutically effective dose is given once or twice a day, preferably twice a day. Examples of dosages for the compounds of formula 1, e.g., compounds 2-5, include but are not limited to: about 10 to about 500 mg once per day, about 10 to about 500 mg twice a day, about 10 mg to about 200 mg twice a day, about 50 mg to about 200 mg administered twice a day, or about 100 mg administered twice a day.

The patient can be continued on the compounds of formula 1, e.g., compounds 2-5, for as long as the condition persists, usually for the remainder of life, at the same dose that was administered initially, or, the dose can be adjusted depending on the progression of the disorder in the judgment of a skilled clinician. Doses may also be adjusted in view of side effects, which may become apparent after prolonged treatment or as a consequence of co-morbidities of the patient (in which case the dose can be reduced and the patient can be continued on the reduced dose). In some instances drug holidays may also be implemented by a physician, as warranted by the response of individual patients.

Progression-modifying treatment may be initiated in a subject after diagnosis of PD by its conventional symptoms, preferentially as soon as possible, or as a prophylactic treatment after detection of one of the disease causing mutations for PD by a suitable genetic test around the expected age of onset. Progression of the disease can be assessed by the Universal Parkinson's Disease Rating Scale (UPDRS) [S. Fahn, C. D. Marsden, D. B. Caine, M. Goldstein, eds.: Recent Developments in Parkinson's Disease, Vol. 2. Florham Park, N.J.]. If prophylactic treatment is indicated, expected ages of onset will take into account the average age of onset and its variance in carriers of the respective mutation in the general population, or preferentially the typical ages of onset generally encountered within the family of the subject. Prophylactic treatment is particularly indicated for heterozygous carriers of a mutation of LRRK2, e.g. the G2019S mutation.

Human patients diagnosed with PD or a similar Parkinsonian syndrome at any stage can be treated either solely with compounds of the formula 1, e.g., compounds 2-5, or in combination with other established treatment regimen, including but not limited to L-DOPA and other dopaminergic agents, dopamine receptor agonists, inhibitors of monoaminoxidase (e.g. selegilin), agents to improve mitochondrial respiration (e.g. Coenzyme Q10), or radical scavenging compounds. The compounds of the formula 1, e.g., compounds 2-5, are preferentially given as oral dosages of 0.1-10 mg/kg, either once or twice daily. Higher and more frequent dosing is preferred for treatment of PD in more advanced stages, while the lower and less frequent doses can be employed at an early stage of the disease, or in a prophylactic mode in disease gene carriers

The compounds of the formula 1, e.g., compounds 2-5, can also be administered by alternate routes, such as subcutaneously, parenterally, transdermally, or by nasal sprays.

The pharmaceutical compositions for oral dosage forms of compounds of the formula 1, e.g., compounds 2-5, may include a variety of inactive adjuvant substances in tablets or capsules, to aid the dissolution of the compounds or modulate the timing of their release (e.g. in extended release formulations). Such ingredients may include but are not limited to high molecular weight polyethylene glycols or polyvinyl pyrrolidones (Povidone), which may preferably be formulated with compounds of the formula 1, e.g., compounds 2-5, in solid dispersions to adjust gastrointestinal release and/or dissolution rate. Compounds of the formula 1, e.g., compounds 2-5, may also be administered orally in form of solutions containing GRAS (Generally Regarded As Safe) vehicle components to aid dissolution, including but not limited to low molecular weight polyethylene glycols (PEGs), polyvinyl pyrrolidones, sorbitol, mannitol and similar polyhydroxylated compounds, carboxymethyl cellulose, dextranes, etc.

Compounds of the formula 1, e.g., compounds 2-5, can advantageously be administered in salt form to aid their dissolution and resorption. Pharmaceutically acceptable salts include, but are not limited to, chlorides, sulfates, phosphates, tosylates, besylates etc.

In another aspect of the present invention, pharmaceutical compositions are provided, which comprise any one of the compounds of the formula 1, e.g., compounds 2-5, (or a prodrug, pharmaceutically acceptable salt or other pharmaceutically acceptable derivative thereof), and optionally comprise a pharmaceutically acceptable carrier. After formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the drug exposure deemed desirable. In certain embodiments, the compounds of the invention may be administered at dosage levels of about 0.1 mg/kg to about 20 mg/kg, or from about 1 mg/kg to about 10 mg/kg of subject body weight per day, one or more times a day, to obtain the desired therapeutic effect. It will also be appreciated that dosages smaller than 0.1 mg/kg or greater than 20 mg/kg can be administered to a subject. In certain embodiments, compounds are administered orally or parenterally.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), cremaphor, glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

In order to maximize drug exposure, it is often desirable to adjust the absorption of the drug by the use of a liquid suspension or crystalline or amorphous material with different water solubility properties. The rate of absorption of the drug then depends upon its rate of dissolution that, in turn, may depend upon crystal size and crystalline form.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinyl pyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, and (j) dissolution rate enhancers like high molecular weight polyethylene glycols or polyvinyl pyrrolidone in physical mixtures or in form of solid dispersions. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions, which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Additionally, the present invention contemplates the use of transdermal patches, which have the added advantage of providing controlled delivery of a compound to the body. Such dosage forms are made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Inhibition of LRRK2 by Compounds of the Invention

LRRK2 is a large protein of about 240 kDa molecular weight, with multiple regulatory domains accounting for an unusually complex regulatory behavior [Mata et al., Trends Neurosci. 29, 286-293 (2006)]. Of note is the dependence on an inhibitory GTPase domain [Ito et al., Biochemistry 46, 1380-1388 (2007); Guo et al., Exp. Cell. Res. 313, 3658-3670 (2007)], where several mutations can cause a pathological increase in kinase activity in an indirect way [Lewis et al., Biochem. Biophys. Res. Commun. 357, 668-671 (2007), Li et al., J. Neurochem. 103, 238-247 (2007)]. The physiological substrates are not yet known, but moesin was identified as a satisfactory substrate in vitro [Jaleel et al., Biochem. J. 405, 307-317 (2007)], and a peptide containing an apparent phosphorylation consensus sequence was derived (LRRKtide) for assay and screening purposes. The activity of LRRK2, however, is rather low in comparison with other well-known kinases.

In the following a method to inhibit the kinase activity of LRRK2, and to determine the potency of an inhibitor, is described, which is particularly suited for low level kinase activities, or compounds of very high potencies requiring very low kinase concentrations to minimize receptor limiting behavior. The assay is conducted according to the Adapta® assay protocol (Invitrogen, published at www.invitroaen.comisite/uslen/horne/Products-and-Services/Applications/Drug-Discovery/Target-and-Lead-Identifaction-and-Validation/KinaseBiology/Kinase-Activity-Assays/Adapta-Universal-Kinase-Assay.html as publication PV4873 and PV4881, respectively, herein incorporated in its entirety by reference) in two stages, the phosphotransferase (kinase) reaction with LRRKtide as substrate, followed by the non-radiometric detection of the ADP formed as a product in the phosphotransferase reaction. In the first step, the recombinantly expressed LRRK2 protein fragment containing relevant regulatory domains [Jaleel et al., Biochem. J. 405, 307-317 (2007)] at a concentration of about 8 nanomole/L is incubated with 200 microM LRRKtide for 1 hour at ambient temperature in the presence of 10 mM MgCl₂ and 100 microM ATP. The reaction is stopped by addition of EDTA, with about 20% of the ATP converted to ADP, within the linear range of the assay. The concentration of ADP is then determined by adding a complex of fluorophore-linked ADP (Alexa Fluor® 647) with a Europium-labelled anti-ADP antibody, emitting a maximal acceptor/donor fluorescent emission ratio at 665 nm/615 nm (FRET-signal). The ADP formed in the assay serves to displace the fluorophore labeled ADP tracer from its antibody, thereby reducing the FRET-signal. To express the amount of ADP in percent of ATP conversion, a standard curve is established from FRET signals of ADP concentrations ranging from 0 to 100 microM ADP. The standard curve is fitted to a sigmoidal binding model appropriate for single binding site displacement of tracer-ADP from its antibody by the unlabelled ADP. To determine the potency of a compound of the general formula 1, the assay is conducted in the presence of varying concentrations of inhibitor, preferentially ranging from 0.1 nanomolar to 1 micromolar in half-logarithmic steps (e.g. 0.1 nM, 0.3 nM, 1 nM, 3 nM, 10 nM, 30 nM, 100 nM, 300 nM, 1000 nM). To determine percent inhibition, control kinase assays are run in the absence of both ATP and inhibitor, simulating complete inhibition since no ADP is formed (100% inhibition control), and in the absence of the inhibitor only, to establish the 0% inhibition control. ADP conversion data of the assays containing varying inhibitor concentrations as a measure of relative kinase activity are modeled to a sigmoidal fit with the 0% and 100% control readings set as top and bottom of the curve. The respective IC₅₀ value is the concentration where the sigmoidal curve intersects with the 50% inhibition level.

It is easily appreciated by anyone skilled in the art that many other ways to assay a protein kinase exist to identify inhibitors and determine their potencies. Different substrates may be used, peptides or whole proteins, which may be natural or artificial substrates. Also, the autophosphorylation of LRRK2 itself can be used as a measure of activity. Assays can be conducted at different concentrations of ATP, which may reflect physiological levels, or may prove advantageous with a particular assay technology. Other assay components may include co-substrates or regulatory molecules, including but not limited to GTP or non-hydrolysable analogs thereof. LRRK2 assays may contain full-length protein, or an active fragment thereof, or various fusion protein constructs, e.g. containing tags commonly used for the convenience of purification, or may make use of mutants of LRRK2, preferably those which cause PD.

It is also readily appreciated that a variety of read-out technologies exist, including but not limited to incorporation of radioactive phosphate into a substrate peptide or protein of LRRK2 followed by recovery of the peptide on a suitable substrate (e.g. phosphocellulose paper), or immunochemical detection and quantification of a substrate phosphoepitope by Western-blotting or various implementations of ELISA formats.

### Inhibition of Neurodegeneration by Compounds of the Invention in a Model Organism

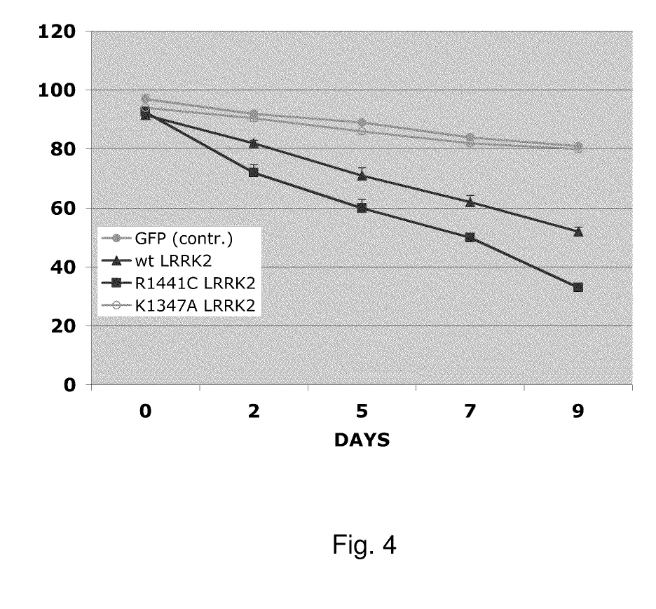

The anti-neurodegenerative activity of compounds of the general formula 1 can be assessed most conveniently in a transgenic C. elegans model to determine an active tissue concentration. C. elegans worms are transfected with a mutant or wildtype human LRRK2 construct under the control of the dopamine transport-1 promoter P-dat1, which is known in the art to restrict expression exclusively to dopaminergic neurons. Of the total of 959 cells that make up the whole organism 8 are known to be dopaminergic neurons. To identify the affected neurons and monitor their structural integrity during the experiments it is advantageous to co-transfect with a GFP (green fluorescent protein) reporter under the control of the same promoter, which renders specifically the transfected neurons fluorescent. Dopaminergic neurodegeneration can then be quantified in form of the loss of total fluorescent signal from a group of worms. In the presence of wt human LRRK2 the loss of neurons is greatly enhanced over controls not transfected with LRRK2 over the course of a few days (FIG. 4), and further amplified in the presence of a mutant LRRK2, e.g. the pathogenic R1441C mutant form. Notably, introducing the artificial mutant K1347A designed to eliminate kinase activity does not have any effect on dopaminergic neurodegeneration (FIG. 4), demonstrating that the kinase activity is pathogenetically relevant, and enhanced over wildtype by PD-associated mutations within the context of the model organism.

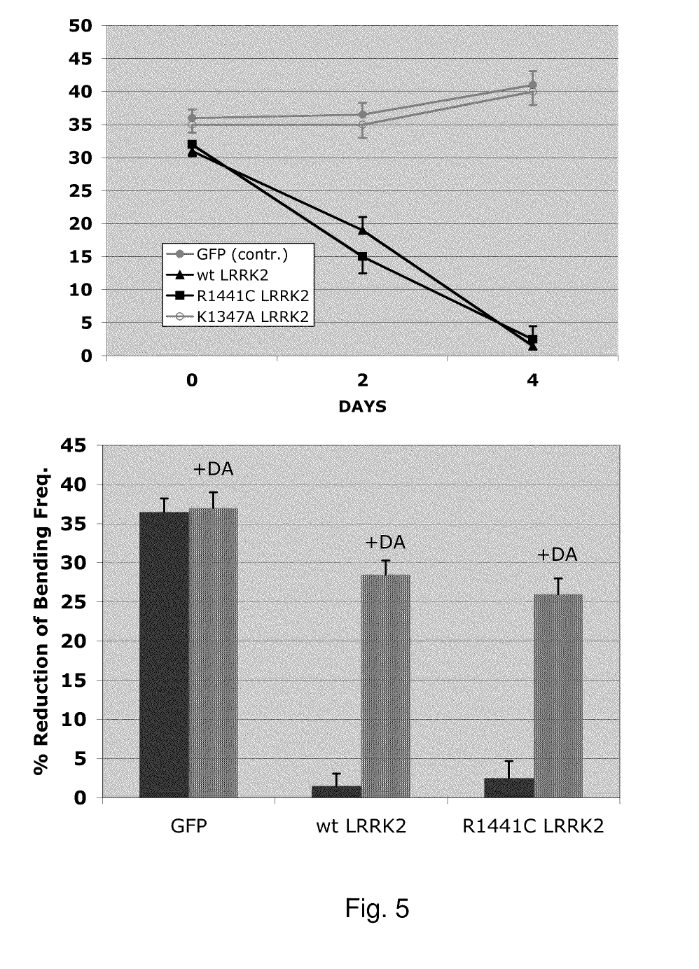

Dopaminergic neurons in C. elegans are involved in food sensing behavior such that in the absence of food worms move more rapidly, as assessed by determining their bending frequency on an agar plate not supplied with food. In an established assay the animals are then exposed to food, and the difference in motility is recorded in the form of bending frequency [Sawin et al., Neuron 26, 619-631 (2000)]. When placed on an agar plate previously coated with a lawn of the E. coli bacterial strain OP50 (prepared as described in: Maintenance of C. elegans, by T. Steiernagle in C. elegans: A Practical Approach, edited by I. A. Hope (1999), Oxford University Press) as a food source, bending frequency is significantly reduced by 35-40% relative to absence of food (FIG. 5). In approximate synchrony with the dopaminergic neurodegeneration this slowing response is essentially eliminated after 4 days. Once again, the kinase dead mutant K1347A does not cause this effect. The slowing response can be reconstituted to a substantial degree by supplementation with dopamine, confirming that the phenotype is due to loss of dopaminergic input (FIG. 5).

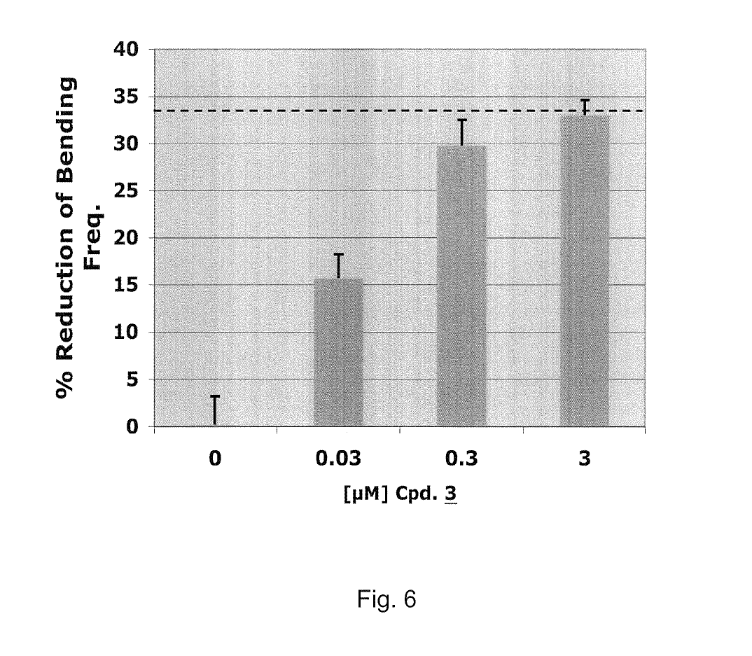

The clear-cut functional phenotype of this model can be used to determine the efficacy of LRRK2 kinase inhibitors and the accurate determination of their potency in a biological context. Compounds of the invention are effective to preserve the dopamine-dependent slowing response at concentrations which are in good agreement with the potency determined in the in vitro kinase assay, as demonstrated for the preferred compound 3 (FIG. 6). A compound of the general formula 1 is added to the bacterial suspension applied as food source for C. elegans worms in the larval stage at concentrations ranging from 3 nM to 3 μM. Plates are spiked with the same concentration to maintain a constant exposure of the organism on the surface, and the feeding suspension is exchanged daily. After reaching adulthood at 4 days of age, worms are briefly placed on an agar plate without food to determine their bending frequency, and then supplied with the feeding suspension to determine the drop in bending frequency. The potency is expressed as the half-maximal restoration of the slowing response. The compound of the formula 3 inhibits the slowing response after food exposure half-maximally at 30 nM.

### Inhibition of the Motor Phenotype of Mutant LRRK2 Transgenic Mice

The anti-neurodegenerative activity of compounds of the general formula 1 can be assessed in vivo under clinically relevant circumstances in a mutant R1441G LRRK2 transgenic mouse model [Li et al., Nat. Neurosci. 12, 826-828 (2009)] which shows a dopamine-deficient motor phenotype in the absence of other transgenes which could complicate the interpretation. In this model a motility impairment, as assessed by a simple cylinder test counting exploratory rearing behavior of mice within a fixed time interval [Baskin et al., J. Neurosci. Methods 129, 87-93 (2003)], becomes apparent at six months of age, and becomes severe at one year of age with an 80% reduction of rearing. The immobility reflected in this impairment measure is equivalent to the hypo- and akinesia of PD.

Compounds of the general formula 1, as exemplified by the compound of the formula 3, retard this decline with regular oral administration. Depending on whether a compound of the general formula 1 is administered as a solution containing polyethylene glycols, or as a suspension of solid compound in an aqueous vehicle, doses of 2.5 to 10 mg/kg twice daily initiated at an age of 6 months reduce the functional decline significantly relative to a vehicle control group after 3-4 months of dosing.

### EXAMPLES

The invention is further illustrated by the following examples, which should not be construed as further limiting. The practice of the present invention will employ, unless otherwise indicated, conventional techniques of organic synthesis, cell biology, cell culture, molecular biology, transgenic biology, microbiology and immunology, which are within the skill of the art.

### Example 1

### Preparation of Compounds of the General Formula I

Compounds of the formula 1 can be prepared from the compound 5 as a common precursor, which is prepared according to WO97/05140, which is incorporated herein by reference in its entirety. The lactams 2 and 3 are obtained by aminolysis of ester 5 with either methylamine or anhydrous ammonia, respectively, at elevated temperatures. Dioxane or tetrahydrofurane are suitable solvents; alternatively, methylamine or ammonia can be used as solvents themselves in pressurized vessels. Solid potassium cyanide can be used as a highly effective catalyst, causing the aminolysis with methylamine as reactant and solvent to occur at room temperature with minimal formation of side products, and thus improving yield and facilitating purification greatly.

The transformation of the lactams 2 and 3 into imides can generally be achieved by oxidation with a CrO₃/pyridine complex in methylene chloride, as described in WO97/05140. Alternatively, this oxidation can also be performed with 5 prior to aminolysis.

Conversion of Compound 5 into Compound 3:

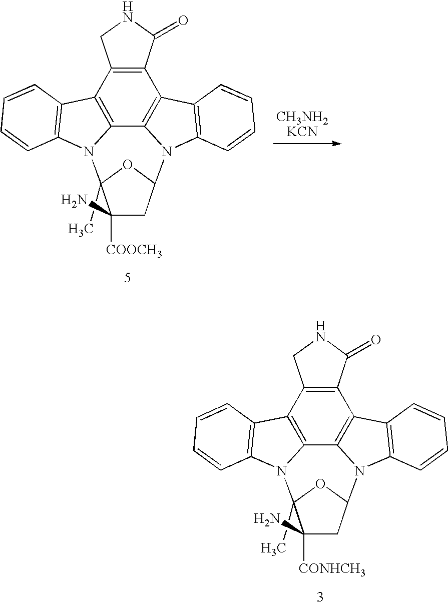

Compound 5, possessing an absolute configuration analogous to natural K252a [Fredenhagen and Peter, Tetrahedron 52, 1235-1238 (1996)], is prepared according to WO97/05140, incorporated herein by reference, as a methylene chloride adduct (86% in pure 5). A mixture of 60.5 mg (0.112 mmole) of 5 and 15 mg KCN is placed into a pressure flask, which 5 ml methyl amine is condensed into at −78° C. The mixture is dissolved by warming up to room temperature and is stirred for 110 hrs under exclusion of light, whereafter according to TLC (silica gel, methylene chloride/methanol 95:5) the starting material (R_{f}=0.28) completely converts into the product 3 (R_{f}=0.25). The solvent is allowed to evaporate, and the colorless solid residue is chromatographed on a 1.5×20 cm silica gel column with methylene chloride/methanol 96:4 as eluent. After evaporation of the solvent 49 mg (81%) of >99.5% (¹H-NMR) pure 3 is obtained.

¹H-NMR (300 MHz, DMSO-d₆): ∂=1.95 (1H, dd, furanoside —CH₂—); 2.11 (3H, s, CH₃); 2.80 (3H, d, CONH—CH₃); 3.35 (1H, m, furanoside —CH₂—, partially obscured by H₂O signal); 5.01 (2H, dd, lactam —CH₂—NH—CO); 7.03 (1H, m, glycosidic —O—CH—N—); 7.27 (1H, t, arom.H); 7.38 (1H, t, arom.H); 7.49 (2H, m, arom.H); 7.85 (1H, d, arom.H); 8.07 (1H, d, arom.H); 8.23 (1H, d, arom.H); 8.32 (1H, m, CO—NH—CH₃); 8.64 (1H, bs, lactam —NH—CO—); 9.22 (1H, d, arom.H). MS (ESI) m/e 466 [M+H]⁺

Conversion of 5 into Compound 2

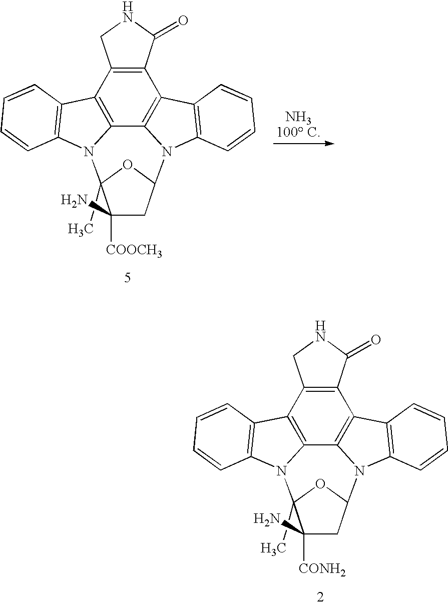

56 mg (0.103 mmole) of the solvate of 5 (86%) with methylene chloride is dissolved in 5 ml dioxane and placed into a glass pressure bomb. 5 ml of anhydrous ammonia is condensed into the vessel at −78° C. The mixture is heated in the pressure bomb under stirring to 100° C. for 24 hrs. Ammonia is allowed to evaporate slowly at room temperature, and the remaining deep yellow solution is evaporated in vacuo to dryness. The resulting yellow solid is resuspended in a few ml methylene chloride/methanol 9:1. Undissolved material is removed by filtration, and the resulting solution is filtered through a short silica gel column with some more methylene chloride/methanol 9:1. The filtrate is evaporated in vacuo to yield 27 mg of yellow crude product, which is further purified by flash chromatography on a 1.5×20 cm silica gel column with methylene chloride/methanol 95:5 as eluent. After evaporation of the fractions containing pure product by TLC (silica gel; methylene chloride/methanol 95:5, R_{f}=0.21) 6.8 mg (14%) of ≧98% pure (¹H-NMR) compound 2 is obtained.

¹H-NMR (300 MHz, DMSO-d₆): ∂=1.94 (1H, dd, furanoside —CH₂—); 2.20 (3H, s, CH₃); 3.22 (1H, m, furanoside —CH₂—, partially obscured by H₂O signal); 5.01 (2H, dd, lactam —CH₂—NH—CO); 7.03 (1H, dd, glycosidic —O—CH—N—); 7.27 (1H, t, arom.H); 7.37 (1H, t, arom.H); 7.48 (2H, m, arom.H); 7.63 (1H, bs, CO—NH₂); 7.79 (1H, bs, CO—NH₂); 7.86 (1H, d, arom.H); 8.07 (1H, d, arom.H); 8.23 (1H, d, arom.H); 8.64 (1H, bs, lactam —NH—CO—); 9.21 (1H, d, arom.H). MS (ESI) m/e 452 [M+H]⁺

Conversion of 5 into Compound 13

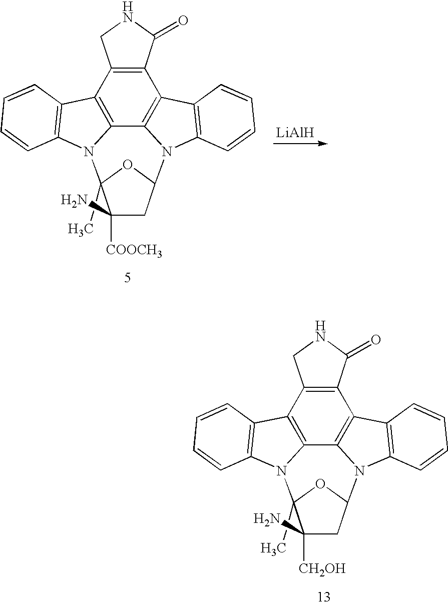

35 mg (0.86 mole) of lithium aluminum hydride was added to an ice-cooled solution of 250 mg (0.43 mmole) compound 5 in 5 ml dry THF and stirred for 2 hrs after warming to room temperature. Excess hydride was quenched with methanol, and the mixture was clarified by filtration over a glass filter. The filtrate was distributed into dichloromethane/water and the organic phase was washed until pH was neutral. The organic phase was dried over sodium sulfate and the solvent was evaporated to yield a solid which was purified by flash chromatography over silica gel with dichloromethane/methanol 95:5 as eluent. Evaporation of product fractions yielded 185 mg (74%) of product 13 as a yellowish solid.

MS (m/e): 439 [M⁺+1]; 312.

Conversion of 3 into Compound 14

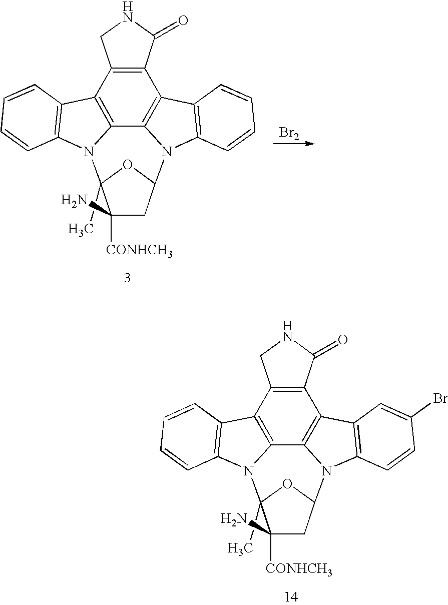

66 mg (0.142 mmole) of compound 3 was dissolved in 2 ml pyridine, and 17 microL (53 mg, or 0.33 mmole) of bromine was added with cooling on ice. The solution was stirred for 2 hrs, and then warmed to room temperature. The mixture was distributed into dichloromethane/5% sodium thiosulfate solution and the organic phase was washed twice with water. After drying over sodium sulfate, the organic phase was evaporated, and the remaining residue was prepurified by flash chromatography on silica gel with dichloromethane/methanol 95:5. The light brown colored crude product was further purified by preparative HPLC with the same eluent to remove unreacted starting material. 32 mg (42%) of product 14 was obtained as a light brown heavy crystal mass.

MS (m/e): 544, 546 (M++1); 390, 392.

### Example 2

### Inhibition of Wildtype Human LRRK2 by Compound 3

Duplicate assay mixtures are set up each in a 10 μL volume containing 17 ng (8 nM) of human LRRK2 protein fragment encompassing amino acids 1326 to 2527, obtained as a recombinant GST fusion protein [according to Jaleel et al., Biochem. J. 405, 307-317 (2007)], 200 μM LRRKtide (Arg-Leu-Gly-Arg-Asp-Lys-Tyr-Lys-Thr-Leu-Arg-Gln-Ile-Arg-Gln peptide), 25 mM Tris, pH 8.2, 5 mM MgCl₂, 0.5 mM EGTA, 100 μM ATP, 0.005% Brij-35, 1% DMSO, and compound 3 in a series of concentrations (OnM, 0.1 nM, 0.3 nM, 1 nM, 3 nM, 10 nM, 30 nM, 100 nM, 300 nM, 1000 nM). After incubation for 1 hr at ambient temperature, 5 μL of the Adapta® Assay Detection Mix (Invitrogen) is added, containing 30 mM EDTA to stop the kinase reaction, 30 nM of the Eu-labelled anti-ADP antibody, and the AlexaFluor°-ADP conjugate. A series of controls is incubated on the same plate with (i) kinase inactivated by EDTA, and (ii) mixtures containing incrementally increased ADP concentrations from 0 to 100 μM, and inversely decreased ATP concentrations from 100 to 0 μM, to establish a standard curve delimited by ADP concentrations corresponding to no conversion and complete conversion of ATP in the assay mixture. The data are fitted to a sigmoidal binding model with the 0% and 100% conversion data points as top and bottom of the curve (Prism Graphpad).

The assays conducted in a 384 well microplate are then read out in a fluorescence microplate reader to establish the ratio of emissions at 665 nm (ADP-tracer) and at 615 nm (Eu-antibody) as a measure of ADP concentration by virtue of Fluorescence Resonance Energy Transfer (FRET). The conversion in % of ATP into ADP by the kinase reaction in each well is determined from the ADP/ATP standard curve, and means are formed from each duplicate assay well. The resultant mean conversion ratios for the kinase assays containing increasing concentrations of inhibitor are fitted to a sigmoidal binding model (Graphpad), with the assay containing no inhibitor (<40% ATP conversion for linearity) taken as the 100% activity control (top), and the control assay with the kinase inhibited by excess EDTA as the 0% activity control (bottom).

The IC₅₀ is determined by the intersection of the fitted curve with the 50% activity measure.

### Example 3

### Inhibition of G2019S Mutant LRRK2 by Compound 3

Inhibition is determined in the same way as in example 2, except that due to the higher specific activity 0.5 ng (2.5 nM) of the mutant aa1326-2527 kinase fragment are used in the assays

All patent and non-patent publications cited in this specification are indicative of the level of skill of those skilled in the art to which this invention pertains. All these publications and patent applications are herein incorporated by reference to the same extent as if each individual publication or patent application was specifically and individually indicated to be incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### Example 4

### Prevention of Neurodegeneration in LRRK2 transgenic C. elegans by Compound 3

C. elegans lines (strain MT1898) stably transfected with the green fluorescent protein (GFP) and the wildtype or mutant R1441C LRRK2, both under the control of the dopamine transporter promoter Pdat-1 [Nass et al., Proc. Natl. Acad. Sci. USA 99, 3264-3269 (2002)], were generated by induction of chromosomal integration with trimethylpsoralen and 365 nm UV irradiation in C. elegans precursor lines stably transfected with both gene constructs in the episomal vector pCEP4 [Guo et al., Exp. Cell Res. 313, 3658-3670 (2007)], followed by outcrossing with the N2 strain for three times. Worms were maintained at room temperature on agar plates supplemented with nematode growth medium (NGM) as described [Hope, 1999. C. elegans: a practical approach. Oxford University Press, New York] and fed with a bacterial suspension of the OP50 E. coli strain. Age synchronization was achieved by collecting newly hatched animals within a time window of a few hours and plating on NGM plates for development through larval stages day 1-4, followed by adult stages day 1-4 for the assays described here.

Degeneration of dopaminergic neurons in transgenic C. elegans was quantified as described before [Berkowitz et al., J. Vis. Exp. 17, 2008)] by averaging the numbers of the four GFP fluorescent in the head region of 30 animals. The remaining neurons in the LRRK2 transgenic worms at any age or after exposure to varying concentrations of compound 3 throughout the larval and adult stages were expressed as a percentage of neurons in worms transgenic with GFP only at the same age, or in vehicle treated worms of the same genotype, respectively. Comparison between groups was by one-way ANOVA.

The functional consequence of dopaminergic neurodegeneration was assayed using the food-sensing response resulting in a slowing of motility after food exposure as assessed by the frequency of body bending within 20 second intervals in groups of 10 animals [Sawin et al., Neuron 26, 619-631 (2000)]. Synchronized worms were grown through the larval stages up to adult stage day 4. Prior to the assay worms were transferred from their food containing plates to NGM plates without food, and then replated on agar inoculated previously with or without OP50 as a food source. The difference in bending frequency in plates with or without food was averaged, and the slowing response in food containing plates relative to those lacking food was expressed in percentage terms and compared by one-way ANOVA.

Treatment with compound 3 was effected by spiking both the NBM supplemented agar and the and the bacterial feeder suspension with varying concentrations of compound 3, including a drug-free control, in the presence of a final concentration of 0.5% DMSO throughout the larval and adult growth stages up to the assay. The difference in the food-induced slowing response between wild-type or mutant LRRK2 transgenic worms treated with varying concentrations of compound 3 was compared to vehicle treated worms (0.5% DMSO) as well as to GFP-only transgenic worms as a control for complete (100%) efficacy.

### Example 5

### Treatment of R1441G LRRK2 Transgenic Mice with Compound 3

The hemizygous R1441G LRRK2 transgenic mouse line as described in Li et al., Nat. Neurosci. 12, 826-828 (2009) are obtained from Jackson laboratories. Three cohorts of transgenic mice and two cohorts of non-transgenic littermates, each containing 20 animals age-matched within a 4 week window are subjected to the cylinder test as adapted to mice according to [Baskin et al., J. Neurosci. Methods 129, 87-93 (2003)] beginning at an average age of 5 months in monthly intervals. The cylinder test is performed in a translucent cylinder 10 cm in diameter with mice placed individually into the cylinder and the number of rearings against the cylinder wall recorded within 5 minute intervals as a measure of mobility. Rearings are averaged within a cohort and variances expressed as standard errors of means (S.E.M.). Twice daily oral dosing is initiated at an age (about 6 months) when the transgenic cohorts are beginning to show a statistically significant performance impairment relative to age-matched non-transgenic litter mates, indicating the onset of disease progression. The three transgenic cohorts are dosed with vehicle (water), 5 mg/kg b.i.d., and 10 mg/kg b.i.d. of a highly dispersed amorphous form of compound 3 suspended in water, respectively. Of the non-transgenic control cohorts one receives water to provide a reference for the onset of impairment and baseline effects of compound 3, while the other is dosed like the transgenic 10 mg/kg treatment cohort to control for baseline effects of compound 3 on the performance of mice without pathology. Dosing is continued for 3-6 months until one or both of the treated transgenic lines reveal a statistically significant better performance, as assessed by one-way ANOVA, relative to the vehicle treated transgenic cohort.

TABLE 1
Specificity of representative LRRK2 inhibitors of the general formula 1
against selected kinases at 100 μM ATP.

| Cpd. | R₁ | R₂ | R₃ | R₄ | LRRK2 | ERK2 | Cdk1 | GSK3 | PKC | PKA |
|---|---|---|---|---|---|---|---|---|---|---|
| 3 | H | CONHCH₃ | H | H | 0.0011 | 0.026 | 0.017 | 0.49 | 0.51 | 0.11 |
| 5 | H | COOCH₃ | H | H | 0.0010 | 0.35 | 0.26 | 35 | 6.5 | 14 |
| K252a | N/A | COOCH₃ | H | H | 0.22 | 0.12 | 0.31 | >10 | 5.0 | 3.4 |
| 13 | H | CH₂OH | H | H | 0.0010 | — | — | — | — | — |
| 14 | H | CONHCH₃ | Br | H | 0.0012 | — | — | — | — | — |
| 15 | H | CONHCH₃ | Br | Br | 0.0015 | — | — | — | — | — |

1. A method for the prevention or treatment of Parkinson's Disease in a subject, comprising administering to a subject in need thereof an effective amount of an indolocarbazole compound of the general formula 1 or a pharmaceutically acceptable salt thereof: 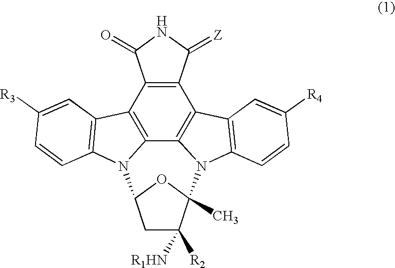
wherein:
R₁ is H, methyl, ethyl, iso-propyl, or acetyl;
R₂ is COOR₅, wherein R₅ is H, methyl, ethyl, isopropyl, cyclopropyl, or —(CH₂)ₙ—Y, wherein Y is OR′, NR′R″, N-morpholinyl, N-piperazinyl, or N′-alkyl-N-piperazinyl, wherein R′ and R″ are independently H or lower n-alkyl and n is 2-6;
or R₂ is CONR₅R₆, wherein R₅ and R₆ are independently H, methyl, ethyl, isopropyl, cyclopropyl, or —(CH₂)ₙ—Y, wherein Y is OR′, NR′R″, N-morpholinyl, N-piperazinyl, or N′-alkyl-N-piperazinyl, wherein R′ and R″ are H or lower n-alkyl, and n is 2-6), or R₅ is any of these definitions, and R₆ is CH₃—(CH₂)ₙ—CO, Ph-CO, or CF₃—CO, wherein n is 0-5;
or R₂ is COX, wherein X is N-morpholinyl, N-piperazinyl, or N′-alkyl-N-piperazinyl;
or R₂ is CH₂OR₅, wherein R₅ is H, methyl, ethyl, isopropyl, cyclopropyl, —(CH₂)ₙ—X, CH₃—(CH₂)ₘ—CO, Ph-CO, or CF₃—CO, wherein X is OR′, NR′R″, N-morpholinyl, N-piperazinyl, or N′-alkyl-N-piperazinyl, wherein R′ and R″ are independently H or lower n-alkyl, n is 2-6, and m is 0-5;
or R₂ is CH₂NR₅R₆, wherein R₅ and R₆ are independently H, methyl, ethyl, isopropyl, cyclopropyl, —(CH₂)ₙ—Y, Ph-CO, or CF₃—CO, wherein Y is OR′, NR′R″, N-morpholinyl, N-piperazinyl, or N′-alkyl-N-piperazinyl, R′ and R″ are independently H or lower n-alkyl, and n is 2-6, or R₅ is any of these definitions, and R₆ is CH₃—(CH₂)ₙ—CO, wherein n is 0-5;
R₃ and R₄ are independently H, F, Cl, Br, methyl, ethyl, propyl, iso-propyl, n-alkyl, OH, OCH₃, O(CH₂)ₙC₃ (n is 1-6), OCH(CH₃)₂, OC(CH₃)₃, CH₃(CH₂)ₙCO (n is 0-5), CF₃—CO, CH₃(CH₂)ₙCOO (n is 0-5), CF₃—COO, or NR₅R₆, wherein R₅ and R₆ are independently H, methyl, ethyl, propyl, iso-propyl, or n-alkyl, or R₅ is any of these definitions, and R₆ is CH₃—CO or CF₃—CO);
or one or both of R₃ and R₄ are independently CH₂—X—(CH₂)ₙCH₃, wherein X═O, S, and n is 0-5;
or one or both of R₃ and R₄ are independently CH₂—NR₅R₆, wherein R₅ and R₆ are independently H, methyl, ethyl, propyl, iso-propyl, n-alkyl, or n-acyl, or R₅ is any of these definitions, and R₆ is CH₃—CO or CF₃—CO); and
Z is either (H, H) or O.

2. The method of claim 1, wherein
R₁ is H, methyl, ethyl, iso-propyl, or acetyl;
R₂ is CH₂OR₅, wherein R₅ is H, methyl, ethyl, isopropyl, cyclopropyl, —(CH₂)ₙ—X, CH₃—(CH₂)ₘ—CO, Ph-CO, or CF₃—CO, wherein X is OR′, NR′R″, N-morpholinyl, N-piperazinyl, or N′-alkyl-N-piperazinyl, R′ and R″ are H or lower n-alkyl, n is 2-6, and m is 0-5;
or R₂ is CH₂NR₅R₆, wherein R₅ and R₆ are independently H, methyl, ethyl, isopropyl, cyclopropyl, or —(CH₂)ₙ—Y, wherein Y is OR′, NR′R″, N-morpholinyl, N-piperazinyl, or N′-alkyl-N-piperazinyl, wherein R′ and R″ are independently H or lower n-alkyl and n is 2-6, or R₅ is any of these definitions, and R₆ is CH₃—(CH₂)ₙ—CO, Ph-CO, or CF₃—CO, wherein n=0-5;
R₃ and R₄ are independently H, F, Cl, Br, methyl, ethyl, propyl, iso-propyl, n-alkyl, OH, OCH₃, O(OH₂)ₙCH₃ (n is 1-6), OCH(CH₃)₂, OC(CH₃)₃, CH₃(CH₂)ₙCO (n is 0-5), CF₃—CO, CH₃(CH₂)ₙCOO (n is 0-5), CF₃—COO, or NR₅R₆, wherein R₅ and R₆ are independently H, methyl, ethyl, propyl, iso-propyl, or n-alkyl, or R₅ is any of these definitions, and R₆ is CH₃—CO or CF₃—CO;
or one or both of R₃ and R₄ are CH₂—X—(CH₂)ₙCH₃. wherein X is O or S, and n is 0-5;
or one or both of R₃ and R₄ are CH₂—NR₅R₆, wherein R₅ and R₆ are independently H, methyl, ethyl, propyl, iso-propyl, n-alkyl, or n-acyl, or R₅ is any of these definitions, and R₆ is CH₃—CO or CF₃—CO), and
Z is (H, H) or O.

3. The method of claim 1, wherein Z is (H, H).

4. The method of claim 1, wherein R₃ and R₄ are H.

5. The method of claim 1, wherein R₁ is H.

6. The method claim 1, wherein R₂ is CONR₅R₆, wherein R₅ and R₆ are each independently H, methyl, ethyl, or isopropyl, or is R₂ is COOR₅, wherein R₅ is H, methyl, ethyl, or isopropyl.

7. The method of claim 1, wherein the compound of the general formula 1 has the formula 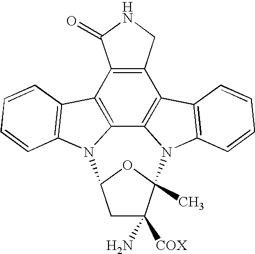
wherein x is NH₂, NHCH₃, N(CH₃)₂, or OCH₃.

8. The method of claim 1, wherein R₁ is H and R₂ is CH₂OH, CH₂OCH₃, or CH₂NR₅R₆, wherein R₅ and R₆ are independently H or methyl, and R₃ and R₄ are H.

9. A method for the prevention or treatment of a synucleopathy involving Lewy body neurodegeneration, comprising administering to a subject in need thereof an effective amount of an indolocarbazole compound of the general formula 1, or a pharmaceutically acceptable salt thereof.

10. The method of claim 9, wherein the synucleopathy involving Lewy body neurodegeneration is idiopathic PD, Dementia with Lewy Bodies (DLB), or familial PD caused by mutations in LRRK2, SNCA (alpha-synuclein), UCHL-1 (ubiquitin carboxyl-terminal hydrolase L1), PRKN (parkin), or PINK-1 (PTEN-induced putative kinase).

11-14. (canceled)

15. A method of inhibiting the activity of wildtype LRRK2 or mutant LRRK2, comprising utilizing a compound of formula 1.

16. A method of treating a disease in a subject, wherein the disease etiology or progression is at least partially mediated by the activity of wildtype LRRK2 or mutant LRRK2, comprising administering to the subject a compound of formula 1.
